(19) United States
(12) Patent Application Publication
(10) Pub. No.: US 2026/0124237 A1
(43) Pub. Date: May 7, 2026

(54) CARTILAGE REGENERATION COMPOSITION

(71) Applicants: KOBAYASHI PHARMACEUTICAL CO., LTD., Osaka-shi, Osaka (JP); KYOTO UNIVERSITY, Kyoto-shi, Kyoto (JP)

(72) Inventors: Yoshiyuki MIZUSHINA, Ibaraki-shi, Osaka (JP); Junya TOGUCHIDA, Kyoto-shi, Kyoto (JP); Yonghui JIN, Kyoto-shi, Kyoto (JP)

(21) Appl. No.: 18/706,335

(22) PCT Filed: Oct. 31, 2022
(86) PCT No.: PCT/JP2022/040710

§ 371 Date: Apr. 30, 2024

(30) Foreign Application Priority Data
Nov. 1, 2021 (JP) 2021-179082

(51) Int. Cl. A61K 31/737; A61K 31/194; A61K 31/728; A61K 36/38; A61K 38/14; A61P 19/02

(52) U.S. Cl. CPC A61K 31/737; A61K 31/194; A61K 31/728; A61K 36/38; A61K 38/14; A61P 19/02

(57) ABSTRACT

The purpose of the present invention is to provide a cartilage regeneration promoter. The cartilage regeneration promoter includes (A) hydroxycitric acid and/or a salt thereof, and (B) a sugar selected from the group consisting of chondroitin sulfate and a salt thereof, hyaluronic acid and a salt thereof, a proteoglycan, and the constituent sugar of the above sugars, and is capable of promoting cartilage regeneration.

### TECHNICAL FIELD

The present invention relates to a cartilage regeneration composition.

### BACKGROUND ART

Joint pain due to cartilage disorder such as osteoarthritis caused by retrograde degeneration of cartilage, cartilage damage due to trauma, or the like is a major factor that restricts daily life activities, and in an aging society, coping with the joint pain is an increasingly important problem. There is no fundamental conservative treatment for cartilage disorder, and oral administration of a nonsteroidal anti-inflammatory drug (NSAID) or intra-articular injection of a steroid is used as a symptomatic treatment for pain. In addition, intra-articular injection of hyaluronic acid, which is an important component of cartilage and one of glycosaminoglycans (GAG), has also been widely performed, and a certain effect on pain has been observed, but the effect on cartilage regeneration is uncertain.

Glucosamine is one of the amino sugars constituting GAG. Glucosamine is contained in the cell walls of crustaceans such as shrimps and crabs, insects such as beetles, and fungi, and is a kind of monosaccharide that is widely present in nature as a constituent unit of chitin. In addition, glucosamine has been studied for its important roles in vivo: for example, glucosamine is present as a constituent sugar of mucopolysaccharides in vivo. Glucosamine not only serves as a component of cartilage and connective tissue. It has been also reported that glucosamine has an anti-inflammatory action via neutrophils and serves as an effective component for arthritis (Patent Document 1).

It is not clear how much orally ingested glucosamine can be involved in the metabolism of articular cartilage. However, attempts have been made to actively utilize glucosamine in foods, quasi-pharmaceutical products, or pharmaceutical products for the purpose of a therapeutic or preventive effect of cartilage disorder. For example, a composition for ameliorating joint pain containing collagen, methylsulfonylmethane, glucosamine, and chondroitin, the composition containing collagen in an intake amount of 2000 mg or more per day has been proposed as a composition for ameliorating joint pain, an agent for ameliorating joint pain, or a food capable of reducing, treating, and preventing symptoms of joint pain such as arthritis (Patent Document 2).

On the other hand, it is known that hydroxycitric acid contained in the pericarp of garcinia suppresses the excess intake of carbohydrates from being stored as body fat, and promotes the consumption of fat. In addition, it has been reported that fat metabolism can be more effectively promoted by using a composition in which caffeine is combined with a garcinia extract (Patent Document 3).

### PRIOR ART DOCUMENT

### Patent Documents

Patent Document 1: Japanese Unexamined Patent Application Publication No. 2004-529860

Patent Document 2: Japanese Patent Laid-open Publication No. 2009-051833

Patent Document 3: Japanese Patent Laid-open Publication No. 2001-258506

### SUMMARY OF THE INVENTION

### Problems to be Solved by the Invention

The effect of glucosamine in treatment or prevention of cartilage disorders has attracted attention, and various glucosamine-containing compositions have been studied, but the effect is not yet at a sufficient level. Therefore, the present inventors have considered that a component having an action to promote cartilage regeneration is expected to have a further effect in treatment or prevention of cartilage disorder. An object of the present invention is to provide a cartilage regeneration promoter having an excellent cartilage regeneration-promoting action.

### Means for Solving the Problem

The present inventors have found that an excellent cartilage regeneration-promoting action is exhibited by combining hydroxycitric acid and/or a salt thereof with a predetermined cartilage component. The present invention has been completed by further conducting studies based on such findings.

That is, the present invention provides inventions of the following aspects.

Item 1. A cartilage regeneration promoter (cartilage regeneration composition) including: (A) hydroxycitric acid and/or a salt of hydroxycitric acid; and (B) a sugar selected from a group consisting of chondroitin sulfate and a salt of chondroitin sulfate, a proteoglycan, hyaluronic acid and a salt of hyaluronic acid, and a constituent sugar of the above sugars.

Item 2. The cartilage regeneration promoter according to Item 1, in which the constituent sugar is an optionally N-acetylated hexosamine selected from a group consisting of glucosamine, N-acetylglucosamine, N-acetylglucosamine sulfate and a salt of N-acetylglucosamine sulfate, galactosamine, N-acetylgalactosamine, and N-acetylgalactosamine sulfate and a salt of N-acetylgalactosamine sulfate.

Item 3. The cartilage regeneration promoter according to Item 1 or 2, in which the component (B) is contained in a total amount of 0.001 to 6 parts by weight with respect to 1 part by weight of the component (A) in a total amount in terms of hydroxycitric acid.

Item 4. The cartilage regeneration promoter according to any one of Items 1 to 3, including a plant extract containing the component (A).

Item 5. The cartilage regeneration promoter according to Item 4, in which the plant extract is a garcinia extract.

Item 6. The cartilage regeneration promoter according to Item 4 or 5, in which the component (B) is contained in a total amount of 0.0015 to 10 parts by weight with respect to 1 part by weight of the plant extract.

Item 7. The cartilage regeneration promoter according to any one of Items 1 to 6, in which the component (B) is chondroitin sulfate.

Item 8. A promoter for differentiation from undifferentiated cells to cartilaginous cells, the promoter including: (A) hydroxycitric acid and/or a salt of hydroxycitric acid; and (B) a sugar selected from a group consisting of chondroitin sulfate and a salt of chondroitin sulfate, a proteoglycan, hyaluronic acid and a salt of hyaluronic acid, and a constituent sugar of the above sugars.

Item 9. A therapeutic agent for knee osteoarthritis including: (A) hydroxycitric acid and/or a salt of hydroxycitric acid; and (B) a sugar selected from a group consisting of chondroitin sulfate and a salt of chondroitin sulfate, a proteoglycan, hyaluronic acid and a salt of hyaluronic acid, and a constituent sugar of the above sugars.

Item 10. A cartilage degradation inhibitor including: (A) hydroxycitric acid and/or a salt of hydroxycitric acid; and (B) a sugar selected from a group consisting of chondroitin sulfate and a salt of chondroitin sulfate, a proteoglycan, hyaluronic acid and a salt of hyaluronic acid, and a constituent sugar of the above sugars.

Item 11. A protective agent for maintaining cartilage thickness including: (A) hydroxycitric acid and/or a salt of hydroxycitric acid; and (B) a sugar selected from a group consisting of chondroitin sulfate and a salt of chondroitin sulfate, a proteoglycan, hyaluronic acid and a salt of hyaluronic acid, and a constituent sugar of the above sugars.

Item 12. An alleviating agent for joint pain, joint uneasiness, and/or joint displeasure, the alleviating agent including: (A) hydroxycitric acid and/or a salt of hydroxycitric acid; and (B) a sugar selected from a group consisting of chondroitin sulfate and a salt of chondroitin sulfate, a proteoglycan, hyaluronic acid and a salt of hyaluronic acid, and a constituent sugar of the above sugars.

Item 13. Use of (A) hydroxycitric acid and/or a salt of hydroxycitric acid; and (B) a sugar selected from a group consisting of chondroitin sulfate and a salt of chondroitin sulfate, a proteoglycan, hyaluronic acid and a salt of hyaluronic acid, and a constituent sugar of the above sugars for producing a cartilage regeneration promoter.

Item 14. Use of (A) hydroxycitric acid and/or a salt of hydroxycitric acid; and (B) a sugar selected from a group consisting of chondroitin sulfate and a salt of chondroitin sulfate, a proteoglycan, hyaluronic acid and a salt of hyaluronic acid, and a constituent sugar of the above sugars for producing a promoter for differentiation from undifferentiated cells to cartilaginous cells, a therapeutic agent for knee osteoarthritis, a cartilage degradation inhibitor, a protective agent for maintaining cartilage thickness, or an alleviating agent for joint pain, joint uneasiness, and/or joint displeasure.

Item 15. Use of a composition including (A) hydroxycitric acid and/or a salt of hydroxycitric acid; and (B) a sugar selected from a group consisting of chondroitin sulfate and a salt of chondroitin sulfate, a proteoglycan, hyaluronic acid and a salt of hyaluronic acid, and a constituent sugar of the above sugars for promoting cartilage regeneration.

Item 16. The use according to Item 15, in which the use is for promoting differentiation from undifferentiated cells to cartilaginous cells, treating knee osteoarthritis, inhibiting cartilage degradation, protection for maintaining cartilage thickness, or alleviating joint pain, joint uneasiness, and/or joint displeasure.

Item 17. A method for regenerating cartilage, the method including a step of administering (A) hydroxycitric acid and/or a salt of hydroxycitric acid; and (B) a sugar selected from a group consisting of chondroitin sulfate and a salt of chondroitin sulfate, a proteoglycan, hyaluronic acid and a salt of hyaluronic acid, and a constituent sugar of the above sugars in an effective amount to an object in need of cartilage regeneration ability.

### Advantages of the Invention

According to the present invention, a cartilage regeneration promoter having an excellent cartilage regeneration-promoting action is provided. Furthermore, according to the present invention, a promoter for differentiation from undifferentiated cells to cartilaginous cells, a therapeutic agent for knee osteoarthritis, a cartilage degradation inhibitor, a protective agent for maintaining cartilage thickness, and an alleviating agent for joint pain, joint uneasiness, and/or joint displeasure are also provided.

### BRIEF DESCRIPTION OF THE DRAWINGS

FIG. 1 shows the results of cartilage matrix production caused by differentiation induction into cartilaginous cells with hydroxycitric acids and/or chondroitin sulfate in three-dimensional culture of human bone marrow-derived mesenchymal stem cells.

FIG. 2 shows the results of cartilage matrix production caused by differentiation induction into cartilaginous cells with hydroxycitric acids and/or glucosamine in three-dimensional culture of human bone marrow-derived mesenchymal stem cells.

FIG. 3 shows the results of cartilage matrix production caused by differentiation induction into cartilaginous cells with hydroxycitric acids and/or hyaluronic acid in three-dimensional culture of human bone marrow-derived mesenchymal stem cells.

FIG. 4 shows the results of cartilage matrix production caused by differentiation induction into cartilaginous cells with hydroxycitric acids and/or a proteoglycan in three-dimensional culture of human bone marrow-derived mesenchymal stem cells.

FIG. 5 shows the results of cartilage matrix production caused by differentiation induction into cartilaginous cells with hydroxycitric acids and/or a predetermined sugar in three-dimensional culture of human bone marrow-derived mesenchymal stem cells.

FIG. 6A shows a stained image of a cross-sectional specimen of a knee femur cartilage prepared from a mouse knee osteoarthritis induction model to which a garcinia extract and/or chondroitin sulfate has been administered.

FIG. 6B shows the result of scoring evaluation of cartilage degeneration based on FIG. 6A.

FIG. 7 shows the measurement result of the amount of a cartilage synthesis marker (CPII) in blood in a mouse knee osteoarthritis induction model to which a garcinia extract and/or chondroitin sulfate has been administered.

FIG. 8 shows the measurement result of the amount of a cartilage degradation marker (C2C) in blood in a mouse knee osteoarthritis induction model to which a garcinia extract and/or chondroitin sulfate has been administered.

### EMBODIMENTS OF THE INVENTION

### 1. Cartilage Regeneration Promoter

The cartilage regeneration promoter of the present invention includes: (A) hydroxycitric acid and/or a salt thereof (hereinafter, also described as “component (A)” or “hydroxycitric acids”); and (B) a sugar selected from a group consisting of chondroitin sulfate and a salt thereof, a proteoglycan, and a constituent sugar of the above sugars (hereinafter, also described as “component (B)” or “predetermined sugar”).

### (A) Hydroxycitric Acids

The cartilage regeneration promoter of the present invention contains hydroxycitric acid and/or a salt thereof as the component (A).

The hydroxycitric acid is an α-hydroxy tribasic acid with two asymmetric centers (1,2-dihydroxypropane-1,2,3-tricarboxylic acid), forming two pairs of diastereoisomers or four different isomers. Specific examples of the hydroxycitric acid include (−) hydroxycitric acid, (+) hydroxycitric acid, (−) allo-hydroxycitric acid, and (+) allo-hydroxycitric acid. These isomers may be used alone, or may be used in combination of two or more thereof. Among these isomers, (−) hydroxycitric acid and (+) allo-hydroxycitric acid are preferable from the viewpoint of obtaining a more preferable cartilage regeneration-promoting effect.

The hydroxycitric acid and the salt thereof are specifically represented by the following general formulas (I) and (II).

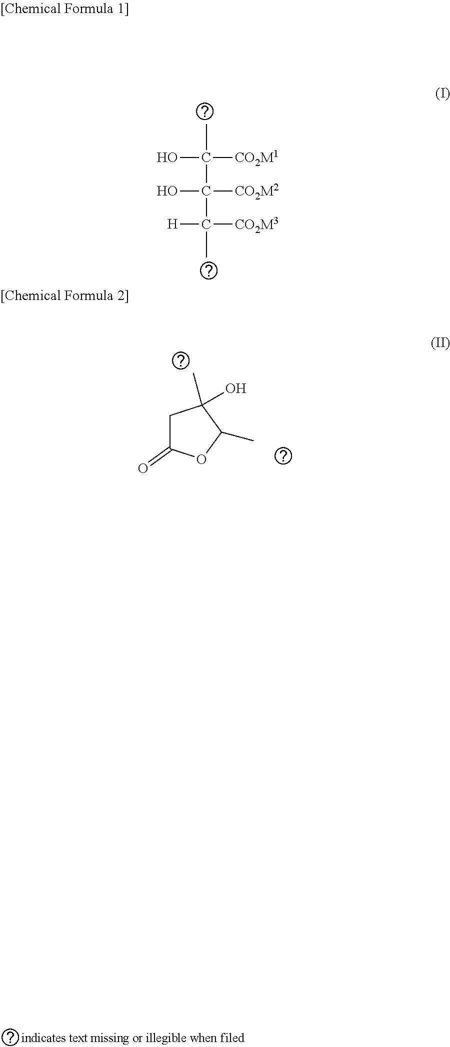

In the present invention, among the hydroxycitric acid and the salt thereof, the compound represented by the formula (I) is also described as “non-lactone form” or “free form”, and the compound represented by the formula (II) (that is, a dehydration condensation cyclized product of the free form) is also described as “lactone form”.

When the compounds represented by the formulas (I) and (II) are hydroxycitric acid, in the formulas (I) and (II), M¹, M², and M³ are all hydrogen atoms.

When the compounds represented by the formulas (I) and (II) are salts of hydroxycitric acid, M¹, M², and M³ in the formula (I) and M¹ and M² in the formula (II) do not simultaneously become a hydrogen atom. The salt of hydroxycitric acid is not particularly limited as long as it is pharmaceutically or cosmetically acceptable.

Specifically, when the compounds represented by the formulas (I) and (II) are salts of hydroxycitric acid, M¹, M², and M³ in the formula (I) and M¹ and M² in the formula (II) each independently represent an alkali metal or an alkaline earth metal, or an organic base. Examples of the alkali metal include potassium and sodium. Examples of the alkaline earth metal include calcium. Examples of the organic base include a monoethanolamine group, a diethanolamine group, a triethanolamine group, an aminomethylpropanol group, and an aminomethylpropanediol group. These salts may be used alone, or may be used in combination of two or more thereof.

In the cartilage regeneration promoter of the present invention, as the hydroxycitric acids, one of the free form and the lactone form may be used, or both may be used in combination. In addition, from among the free form and the lactone form, one may be selected and used, or two or more may be used in combination. Among these free forms and lactone forms of hydroxycitric acids, a free form is preferable from the viewpoint of obtaining a more excellent cartilage regeneration-promoting effect.

In the cartilage regeneration promoter of the present invention, as the hydroxycitric acids, one of the hydroxycitric acid and the salt of hydroxycitric acid may be used, or both may be used in combination. In addition, from among the hydroxycitric acid and the salt of hydroxycitric acid, one may be selected and used, or two or more may be used in combination. Among these hydroxycitric acids and salts thereof, a salt of hydroxycitric acid is preferred, an alkali metal salt and an alkaline earth metal salt are more preferred, an alkaline earth metal salt is further preferred, and a calcium salt is particularly preferred from the viewpoint of obtaining a more excellent cartilage regeneration-promoting effect.

In the cartilage regeneration promoter of the present invention, the hydroxycitric acids may be obtained from natural products or may be chemically synthesized. Examples of the natural product include plants such as the species Garcinia of the genus Garcinia in the family Hypericaceae (specific examples thereof include Garcinia cambogia, Garcinia indica, Garcinia atroviridis, Garcinia mangostana, and Garcinia subelliptica) and Hibiscus L. in the genus Hibiscus in the family Malvaceae, preferably the genus Garcinia, and more preferably Garcinia cambogia. The plant may be produced by cultivation or collected from nature. The part of the plant to be used is not limited as long as it is a part containing the hydroxycitric acids, but preferably includes the pericarp. Methods for obtaining the hydroxycitric acids from plants are conventionally known. The cartilage regeneration promoter of the present invention may contain an isolated and purified product of the hydroxycitric acids from natural products or may contain a crude purified product of the hydroxycitric acids from natural products.

Examples of the crude purified product of the hydroxycitric acids include a processed product of the plant described above, and preferably include a processed product of a pericarp. Specific embodiments of the processed product of the plant described above include a plant dried product, a plant ground product (including raw and dried products), and a plant extract. As the processed product of the plant, a product derived from a single plant may be used, or two or more products derived from different plants may be used in combination. Among these processed products of the plant, a plant extract is preferred.

Among the crude purified product of the processed product of the plant described above, from the viewpoint of obtaining a more excellent cartilage regeneration-promoting effect, a plant extract is preferred, an extract obtained from the genus Garcinia (garcinia extract) (particularly preferably an extract obtained from Garcinia cambogia) and a hibiscus extract obtained from the genus Hibiscus in the family Malvaceae are more preferred, and a garcinia extract is still more preferred. The plant extract may be a squeezed juice, a solvent extract, or a fraction containing the hydroxycitric acids from a solvent extract. The method for obtaining the plant extract is not particularly limited, and for example, the plant extract can be obtained as follows. The plant extract can be prepared, for example, by preparing a pericarp of a plant such as the species Garcinia in a raw state or in a state of a dried product, and holding its size or cutting or pulverizing the pericarp as necessary, followed by performing a conventional extraction method such as solvent extraction and supercritical extraction. Examples of the extraction solvent include water (including warm water and hot water), organic solvents (lower alcohols having 1 to 4 carbon atoms such as methanol, ethanol, n-propanol, isopropanol, and n-butanol; polyhydric alcohols such as propylene glycol and 1,3-butylene glycol; ketones such as acetone; esters such as diethyl ether, dioxane, acetonitrile, and ethyl acetate; xylene, benzene, chloroform, etc.), and a mixture thereof, preferably include water, a lower alcohol, and a mixture thereof, more preferably include heated water such as warm water and hot water, and further preferably include hot water. These solvents may be used singly or in combination of two or more kinds thereof.

Furthermore, examples of the method for obtaining a plant extract containing a salt of hydroxycitric acid by using a raw material plant containing hydroxycitric acid includes: a method of performing an extraction operation in the presence of an alkali metal salt and/or an alkaline earth metal salt (for example, when obtaining a plant extract containing calcium hydroxycitrate, calcium salts such as calcium carbonate, calcium lactate, and calcium eggshell can be mentioned); and a method of obtaining a plant extract containing hydroxycitric acid, and then treating the plant extract with a basic compound of an alkali metal and/or an alkaline earth metal to change hydroxycitric acid in the extract into a salt.

When the obtained plant extract (a squeezed juice, a solvent extract, a fraction containing the hydroxycitric acids from a solvent extract, and the like) is contained in the cartilage regeneration promoter, the plant extract may be in the form of a non-concentrated extract that is not concentrated as it is, in the form of a concentrated liquid soft extract, or in the form of an extract powder obtained by further drying the non-concentrated extract or the soft extract. Examples of the drying treatment include spray drying treatment and freeze-drying treatment.

The amount of the hydroxycitric acids is, for example, 10 wt % or more, preferably 30 wt % or more, more preferably 50 wt % or more, still more preferably 55 wt % or more, in 100 wt % of the crude purified product of the hydroxycitric acids (preferably a processed product of a plant, more preferably a plant extract) in terms of dry weight. The upper limit of the amount of the hydroxycitric acids in 100 wt % in terms of dry weight is not particularly limited, and is, for example, 80 wt % or less or 70 wt % or less.

The method for obtaining the isolated and purified product of the hydroxycitric acids is not particularly limited, and examples thereof include a method of further purifying a fraction containing the hydroxycitric acids from the above-described extract. The purification treatment may be a method in which the hydroxycitric acids are isolated and further purified, and can be performed according to a conventional method. Examples thereof include separation treatment such as chromatography and recrystallization treatment.

The blended amount of the component (A) in the cartilage regeneration promoter of the present invention is not particularly limited, but is, for example, 20 to 99 wt %, preferably 30 to 90 wt %, more preferably 35 to 80 wt %, and still more preferably 38 to 70 wt % in a total amount in terms of hydroxycitric acid.

### (B) Predetermined Sugar

The cartilage regeneration promoter of the present invention includes, as the component (B), a predetermined sugar selected from a group consisting of (B1) chondroitin sulfate and a salt thereof (hereinafter, also described as “component (B1)”), (B2) a proteoglycan (hereinafter, also described as “component (B2)”), (B3) hyaluronic acid and a salt thereof (hereinafter, also described as “component (B3)”), and (B4) a constituent sugar of the above sugars (hereinafter, also described as “component (B4)”).

The chondroitin sulfate as the component (B1) is a compound known as an acidic mucopolysaccharide in which sulfuric acid is bonded to a sugar chain having two sugars as its repeating structural units: N-acetylgalactosamine (referring to N-acetyl-D-galactosamine; hereinafter, the same applies); and glucuronic acid (referring to D-glucuronic acid; hereinafter, the same applies) or iduronic acid (referring to L-iduronic acid; hereinafter, the same applies).

Examples of the chondroitin sulfate include chondroitin sulfate A (the constituent two sugars are glucuronic acid and acetylgalactosamine 4-sulfate), chondroitin sulfate B (the constituent two sugars are iduronic acid 2-sulfate and acetylgalactosamine 4-sulfate.; also called dermatan sulfate), chondroitin sulfate C (the constituent two sugars are glucuronic acid and acetylgalactosamine 6-sulfate), chondroitin sulfate D (the constituent two sugars are glucuronic acid 2-sulfate and acetylgalactosamine 6-sulfate), and chondroitin sulfate E (the constituent two sugars are glucuronic acid and acetylgalactosamine-4,6-disulfate). These chondroitin sulfates may be used alone, or may be used in combination of two or more thereof. The chondroitin sulfate used in the present invention preferably contains at least chondroitin sulfate A.

The salt of chondroitin sulfate is not particularly limited, and examples thereof include alkali metal salts such as a sodium salt and a potassium salt; alkaline earth metal salts such as a calcium salt and a magnesium salt; an ammonium salt; basic amino acid salts such as arginine, lysine, histidine, and ornithine; amine salts such as a monoethanolamine salt and a diethanolamine salt. These salts may be used singly or in combination of two or more kinds thereof.

As the component (B1), one of chondroitin sulfate and a salt of chondroitin sulfate may be used, or both of chondroitin sulfate and a salt of chondroitin sulfate may be used in combination. Among them, chondroitin sulfate is preferable.

The chondroitin sulfate and the salt thereof may be chemically synthesized or may be extracted or purified from a material derived from a natural product. As the chondroitin sulfate or the salt thereof, chondroitin sulfate or a salt thereof commercially available as a reagent may be used.

The method for extracting or purifying and producing chondroitin sulfate or a salt thereof from a material derived from a natural product is not particularly limited, and examples thereof include a method of extraction and purification from a material derived from a natural product by a known method. Examples of the material derived from a natural product for extracting chondroitin sulfate include cartilage of mammals such as pig and cattle; cartilage of birds such as chicken; and cartilage of fish such as salmon, ray, and shark, preferably include cartilage of mammals, and more preferably include porcine cartilage.

The proteoglycan as the component (B2) is a complex of a mucopolysaccharide and a protein. The mucopolysaccharide constituting the proteoglycan may be a polysaccharide including a repeating structure of an optionally N-acetylated hexosamine and glucuronic acid or iduronic acid. Examples of the optionally N-acetylated hexosamine include glucosamine, N-acetylglucosamine, N-acetylglucosamine sulfate and a salt thereof, galactosamine, N-acetylgalactosamine, and N-acetylgalactosamine sulfate and a salt thereof. Examples of the mucopolysaccharide include chondroitin sulfate (acidic mucopolysaccharide described as the component (B1)), heparan sulfate (acidic mucopolysaccharide in which sulfuric acid is bonded to a sugar chain having glucosamine and glucuronic acid or iduronic acid as its repeating structural units), heparin (acidic mucopolysaccharide in which sulfuric acid is bonded to a sugar chain having glucosamine and glucuronic acid or iduronic acid as its repeating structural units), and keratan sulfate (acidic mucopolysaccharide in which sulfuric acid is bonded to a sugar chain having N-acetylglucosamine and galactose as its repeating structural units). Examples of the salt include alkali metal salts such as sodium salts and potassium salts, salts of alkaline earth metal such as calcium and magnesium, and salts of metal such as aluminum.

The proteoglycan may be used singly or in combination of two or more kinds thereof.

The proteoglycan may be chemically synthesized or may be extracted or purified from a material derived from a natural product. As the proteoglycan, proteoglycan commercially available as a reagent may be used.

The method for extracting or purifying and producing proteoglycan from a material derived from a natural product is not particularly limited, and examples thereof include a method of extraction and purification from a material derived from a natural product by a known method. Examples of the material derived from a natural product for extracting proteoglycan include cartilage of mammals such as pig and cattle; cartilage of birds such as chicken; and cartilage of fish such as salmon, ray, and shark, preferably include fish cartilage, and more preferably include salmon cartilage.

The hyaluronic acid as the component (B3) is a compound known as an acidic mucopolysaccharide having a linear sugar chain having two sugars as its repeating structural units: N-acetylglucosamine (referring to N-acetyl-D-glucosamine; hereinafter, the same applies) and glucuronic acid.

The salt of hyaluronic acid is not particularly limited, and examples thereof include alkali metal salts such as a sodium salt and a potassium salt; alkaline earth metal salts such as a calcium salt and a magnesium salt; an ammonium salt; basic amino acid salts such as arginine, lysine, histidine, and ornithine; and amine salts such as a monoethanolamine salt and a diethanolamine salt. These salts may be used singly or in combination of two or more kinds thereof.

As the component (B3), one of hyaluronic acid and a salt of hyaluronic acid may be used, or both of hyaluronic acid and a salt of hyaluronic acid may be used in combination. Among them, hyaluronic acid is preferable.

The hyaluronic acid and the salt thereof may be chemically synthesized or may be extracted or purified from a material derived from a natural product. As the hyaluronic acid or the salt thereof, hyaluronic acid or a salt thereof commercially available as a reagent may be used.

The method for extracting or purifying and producing hyaluronic acid or a salt thereof from a material derived from a natural product is not particularly limited, and examples thereof include: fermentation with microorganisms; and a method of extraction and purification from a material derived from a natural product by a known method. Examples of microorganisms used in the fermentation include lactic acid bacteria and Bacillus subtilis natto. Examples of the material derived from a natural product for extracting hyaluronic acid include cartilage and skin of mammals such as pig and cattle; cockscomb; eye ball and cartilage of fish such as salmon, arabesque greenling, codfish, and shark, and preferably include cockscomb.

The constituent sugar as the component (B4) is not particularly limited as long as it is a constituent sugar of the component (B1), the component (B2), or the component (B3). The component (B4) includes both a sugar chain and a monosaccharide. Among them, the sugar chain is a mucopolysaccharide, which is a constituent sugar chain of the component (B2). Specific examples of the mucopolysaccharide as the component (B4) include polysaccharides described in the component (B2) other than the components (B1) and (B3). The monosaccharide includes glucuronic acid, iduronic acid, and an optionally N-acetylated hexosamine selected from a group consisting of glucosamine, N-acetylglucosamine, N-acetylglucosamine sulfate and a salt thereof, galactosamine, N-acetylgalactosamine, and N-acetylgalactosamine sulfate and a salt thereof. These sugars may be used singly or in combination of two or more kinds thereof. Among these sugars, from the viewpoint of further enhancing the effect of the present invention, an optionally N-acetylated hexosamine is preferable, glucosamine, galactosamine, N-acetylgalactosamine, or N-acetylgalactosamine sulfate and salts thereof are more preferable, and N-acetylgalactosamine sulfate and salts thereof are still more preferable.

The constituent sugar may be chemically synthesized or may be extracted or purified from a material derived from a natural product. As the constituent sugar, sugars commercially available as a reagent may be used.

The method for extracting or purifying and producing the constituent sugar from a material derived from a natural product is not particularly limited, and examples thereof include a method of extraction and purification from a material derived from a natural product by a known method. Examples of the material derived from a natural product for extracting the constituent sugar include cartilage of mammals such as pig and cattle; cartilage of birds such as chicken; and cartilage of fish such as salmon, ray, and shark. Especially for mucopolysaccharides, included is cartilage of birds such as chicken.

In the present invention, as the component (B), only a single type may be used, or a plurality of types may be used in combination among the four types of components: the component (B1), the component (B2), the component (B3), and the component (B4). In the present invention, the component (B1) is preferable from the viewpoint of further enhancing the effect of the present invention.

In the cartilage regeneration promoter of the present invention, the content of the component (B) is not particularly limited, but for example, the component (B) is contained in a total amount of 0.0001 to 6 parts by weight with respect to 1 part by weight of the component (A) in a total amount in terms of hydroxycitric acid. From the viewpoint of further enhancing the effect of the present invention, the content is preferably 0.0002 to 4 parts by weight, and more preferably the following content. When the component (B) is used together with a plant extract containing the component (A), the amount of the component (B) is 0.0001 to 3.6 parts by weight per 1 part by weight of the plant extract (in terms of dry weight). From the viewpoint of further enhancing the effect of the present invention, the amount is preferably 0.0001 to 2.4 parts by weight, more preferably the following content.

### When the Component (B1) is Used:

The amount of the component (B1) with respect to 1 part by weight of the component (A) in a total amount in terms of hydroxycitric acid is preferably 0.001 to 2 parts by weight, more preferably 0.0014 to 1.5 parts by weight, still more preferably 0.005 to 0.5 parts by weight, still more preferably 0.01 to 0.2 parts by weight, still more preferably 0.02 to 0.15 parts by weight, and particularly preferably 0.03 to 0.1 parts by weight (furthermore, the lower limit may be 0.044 parts by weight or more, 0.05 parts by weight or more, or 0.06 parts by weight or more, and the upper limit may be 0.095 parts by weight or less, 0.09 parts by weight or less, or 0.085 parts by weight or less). The preferred amount of the (B1) is applied to both the case of using a plant extract containing (A) and the case of not using the plant extract (for example, a case of using a reagent of hydroxycitric acid and/or a salt thereof as the component (A)).

When the plant extract containing the component (A) is used, the amount of the component (B1) with respect to 1 part by weight of the plant extract (in terms of dry weight) is preferably 0.0008 to 1 parts by weight, more preferably 0.005 to 0.15 parts by weight, still more preferably 0.007 to 0.1 parts by weight, even more preferably 0.02 to 0.07 parts by weight (Furthermore, the lower limit may be 0.025 parts by weight or more, 0.03 parts by weight or more, or 0.03 parts by weight or more, and the upper limit may be 0.08 parts by weight or less, 0.06 parts by weight or less, or 0.05 parts by weight or less).

### When the Component (B2) is Used:

The amount of the component (B2) with respect to 1 part by weight of the component (A) in a total amount in terms of hydroxycitric acid is preferably 0.0003 to 0.4 parts by weight, more preferably 0.0035 to 0.2 parts by weight, still more preferably 0.0015 to 1 part by weight, still more preferably 0.002 to 0.3 parts by weight, and still more preferably 0.01 to 0.05 parts by weight. The preferred amount of the (B2) is applied to both the case of using a plant extract containing (A) and the case of not using the plant extract (for example, a case of using a reagent of hydroxycitric acid and/or a salt thereof as the component (A)).

When the plant extract containing the component (A) is used, the amount of the component (B2) with respect to 1 part by weight of the plant extract (in terms of dry weight) is preferably 0.0002 to 0.5 parts by weight, more preferably 0.0008 to 0.3 parts by weight, still more preferably 0.002 to 0.2 parts by weight, even more preferably 0.008 to 0.13 parts by weight.

### When the Component (B3) is Used:

The amount of the component (B3) with respect to 1 part by weight of the component (A) in a total amount in terms of hydroxycitric acid is preferably 0.0015 to 3 parts by weight in a total amount. The amount of the component (B3) with respect to 1 part by weight of the component (A) in a total amount in terms of hydroxycitric acid is more preferably 0.01 to 2 parts by weight, still more preferably 0.1 to 1 part by weight, still more preferably 0.2 to 0.6 parts by weight in a total amount.

When the plant extract containing the component (A) is used, the amount of the component (B3) with respect to 1 part by weight of the plant extract (in terms of dry weight) is preferably 0.0009 to 1.8 parts by weight, more preferably 0.0009 to 1.2 parts by weight in a total amount.

### When the Component (B4) is Used:

The amount of the component (B4) with respect to 1 part by weight of the component (A) in a total amount in terms of hydroxycitric acid is preferably 0.0015 to 3 parts by weight in a total amount. When the component (B4) is glucosamine, the amount of the component (B4) with respect to 1 part by weight of the component (A) in a total amount in terms of hydroxycitric acid is preferably 0.0015 to 2 parts by weight, more preferably 0.01 to 1 part by weight, still more preferably 0.01 to 0.5 parts by weight, still more preferably 0.04 to 0.3 parts by weight. When the component (B4) is other than glucosamine, the amount of the component (B4) with respect to 1 part by weight of the component (A) in a total amount in terms of hydroxycitric acid is preferably 0.01 to 2 parts by weight, more preferably 0.1 to 1 part by weight, still more preferably 0.2 to 0.6 parts by weight in a total amount.

When the plant extract containing the component (A) is used, the amount of the component (B4) with respect to 1 part by weight of the plant extract (in terms of dry weight) is preferably 0.0009 to 1.8 parts by weight, more preferably 0.0009 to 1.2 parts by weight in a total amount. When the component (B4) is glucosamine, the amount of the component (B4) with respect to 1 part by weight of the plant extract (in terms of dry weight) is preferably 0.001 to 1 part by weight, more preferably 0.006 to 0.3 parts by weight, still more preferably 0.01 to 0.2 parts by weight.

The specific blended amount of the component (B) in the cartilage regeneration promoter of the present invention is not particularly limited, and may be preferably set so that the ratio to the blended amount of the component (A) is the above ratio. Specific examples of the blended amount of the component (B) include 0.01 to 10 wt %, preferably 0.1 to 5 wt %, more preferably 0.3 to 4 wt %, and still more preferably 1 to 3 wt %.

### Other Components

The cartilage regeneration promoter of the present invention optionally contains other components in addition to the component (A) and the component (B) according to the application form as long as the effects of the present invention are not impaired. Examples of such other components include physiologically active substances and additives.

Examples of the physiologically active substance preferably include a component that is effective in ameliorating cartilage or joint, and especially include collagen, type II collagen, non-denatured active type 2 collagen, a collagen peptide, methylsulfonylmethane (MSM), S-adenosylmethionine, creatine, theanine, piperine, maslinic acid, 5-aminolevulinic acid phosphate, cat's-claw, black ginger, bosueliaserata, artichoke, amino acid, vitamin A, vitamin B1, vitamin B2, vitamin B6, vitamin B12, vitamin C, vitamin D2, vitamin D3, vitamin E, vitamin K, folic acid, an elastin peptide derived from bonitos, an imidazole dipeptide, a quercetin glycoside, EPA/DHA derived from krill oil, glucomoringin derived from moringa seed, Japanese knotweed, devil's claw, a production promoter for hyaluronic acid derived from chicken foot (HAS-II), soybean isoflavone, β-cryptoxanthin, bone pep, concentrated whey active protein (CBP), 3-hydroxy-3-methyl butyrate (HMB), calcium bis-3-hydroxy-3-methyl butyrate monohydrate (HMB calcium), calcium maltobionate, calcium, magnesium, zinc, iron, selenium, potassium, estrogen, calcitonin, aspirin, steroidal anti-inflammatory agents, and nonsteroidal anti-inflammatory agents. These physiologically active substances may be used singly or in combination of two or more kinds thereof.

Examples of the additive include excipients, disintegrants, diluents, lubricants, flavoring agents, coloring agents, sweetening agents, corrigents, suspending agents, wetting agents, emulsifying agents, dispersing agents, auxiliaries, preservatives, buffers, binders, stabilizers, extenders, thickeners, pH adjusters, surfactants, coating agents, and nutritional components, which are pharmaceutically or bromatologically acceptable. These additives may be used singly or in combination of two or more kinds thereof.

### Preparation Forms

The form and property of the cartilage regeneration promoter of the present invention are not particularly limited as long as it contains the component (A) and the component (B).

The administration form of the cartilage regeneration promoter of the present invention includes both an oral administration form and a parenteral administration form. Therefore, the cartilage regeneration promoter of the present invention is prepared as an oral preparation, an injection, a drip infusion, a nasal drop, a transdermal absorption agent (external preparation), or the like. Since the cartilage regeneration promoter of the present invention is used for the purpose of regenerating cartilage, it is preferable that the cartilage regeneration promoter is an oral preparation, which can be easily administered (ingested) on a daily and/or continuous basis.

The property of the cartilage regeneration promoter of the present invention may be liquid or solid. Examples of the liquid include a liquid form, a beverage form, an emulsion form, a suspension form, an alcohol form, a syrup form, an elixir form, a soft extract form, and the like. Examples of the solid include a tablet, a pill form, a powder form, a fine granule form, a granule form, a capsule form (including a hard capsule and a soft capsule), a troche form, a chewable form, and the like. When the cartilage regeneration promoter of the present invention is in a solid form, the cartilage regeneration promoter may be a durable form or a sustained or a sustained release form, or may be mixed with water or the like at the time of administration (ingestion).

The cartilage regeneration promoter of the present invention can be used as foods in general, foods with health claims (including foods for specified health uses, foods with nutrient function claims, foods with function claims, and supplements), foods for patients, pharmaceutical products, or quasi-pharmaceutical products. Therefore, the cartilage regeneration promoter of the present invention can be prepared substantially as a food product or a pharmaceutical composition containing the component (A) and the component (B). In particular, from the viewpoint of daily and/or continuous easy intake, use as a supplement is preferable.

### Production Method

The production method of the cartilage regeneration promoter of the present invention may be carried out according to a conventionally known normal preparation procedure using the component (A) and the component (B), and other components blended as necessary, depending on various forms and properties, and intended use.

### Application

The cartilage regeneration promoter of the present invention promotes cartilage regeneration by administration (ingestion). Cartilage regeneration refers to reconstructing deficient or degenerated cartilage tissue with normal cartilage tissue (hyaline cartilage) by synthesizing cartilage, that is, by differentiating undifferentiated cells into cartilaginous cells, growing them, and synthesizing cartilage matrix. Promoting cartilage regeneration refers to promoting the cartilage regeneration, suppressing further degradation of cartilage tissue, and promoting reconstruction of cartilage tissue.

Therefore, the cartilage regeneration promoter of the present invention can be used for a subject in need of cartilage regeneration ability for the purpose of suppressing and/or regenerating wear of cartilage, enhancing the ability to make cartilage, supporting by promoting the production of a cartilage component (specifically, cartilage matrix), and/or maintaining a healthy state of cartilage.

In the cartilage regeneration promoter of the present invention, more specific examples of the above purpose include: helping (that is, supporting) bending and stretching the knee joint (that is, smoothness of knee joint motion), flexibility, mobility, and/or reducing annoyance in walking (in particular, in walking a long distance for a certain period of time), going up and down stairs, taking on or off socks, standing up, sitting down, crouching, picking something on the floor, and/or normal activities.

Animal species as the subject in need of cartilage regeneration ability preferably include mammals, and more specifically, human; pet animals such as dogs and cats; and domesticated animals such as horses and cattle.

The hydroxycitric acids, which are active ingredients of the cartilage regeneration promoter of the present invention, have not only a cartilage synthesis-promoting action but also a cartilage degradation-suppressing action. Thus, in view of such an effect of the present invention, one embodiment of preferable application of the cartilage regeneration promoter of the present invention includes: application in a subject having cartilage disorder (an example of a subject in need of cartilage regeneration ability) for the purpose of treating the cartilage disorder. The treatment of cartilage disorder includes not only complete cure but also reduction of cartilage disorder. The cartilage disorder is not particularly limited as long as it is a disease state caused by deficiency or degeneration of cartilage, and examples thereof include osteoarthritis, traumatic cartilage injury, and arthritis due to various causes. The target cartilage site is not particularly limited, and examples thereof include various joint sites in the body, such as a knee joint, a hip joint, an elbow joint, a shoulder joint, a wrist joint, an ankle joint, and a temporomandibular joint.

The hydroxycitric acids, which are active ingredients of the cartilage regeneration promoter of the present invention, have an excellent cartilage synthesis-promoting action and an excellent cartilage degradation-suppressing action. Thus, in view of such an effect of the present invention, one embodiment of preferable application of the cartilage regeneration promoter of the present invention includes: application in a subject having a risk of cartilage disorder (an example of a subject in need of cartilage regeneration ability) for the purpose of preventing cartilage disorder. Examples of the subject having a risk of cartilage disorder include, in the case of a human, an elderly person who is at risk of osteoarthritis, which is often observed in persons who are 50 years old or older, particularly in persons who are 65 years old or older, sports fans or professional sports players who are at risk of cartilage damage observed in sports injuries, other persons who overuse joints on a daily basis, or persons who cause natural wear of cartilage associated with aging, and preferably include persons who cause natural wear of cartilage associated with aging. When the cartilage regeneration promoter of the present invention is applied to persons who cause natural wear of cartilage associated with aging, the cartilage regeneration promoter of the present invention can be used, utilizing the action of suppressing degradation of cartilage tissue and the action of promoting reconstruction of cartilage tissue, preferably for the purpose of maintaining the thickness of knee cartilage that has become thinner with aging, and reducing knee pain, knee uneasiness, and/or knee displeasure in daily life.

The hydroxycitric acids, which are active ingredients of the cartilage regeneration promoter of the present invention, also have an action of differentiation from undifferentiated cells to cartilaginous cells. In view of such an effect of the present invention, one embodiment of preferable application of the cartilage regeneration promoter of the present invention includes: application in a subject having such a severe cartilage disorder that a defect of cartilage itself (that is, cartilage deficiency) is observed (an example of a subject in need of cartilage regeneration ability) for the purpose of treatment of regenerating cartilage itself. The treatment of severe cartilage disorder includes not only complete cure but also reduction of cartilage disorder. Specific examples of severe cartilage disorder include traumatic cartilage deficiency caused by sports, traffic accidents, or the like.

### Dose

The dose of the cartilage regeneration promoter of the present invention is, for example, 0.1 g/day/60 kg or more, preferably 0.2 g/day/60 kg or more, and more preferably 0.25 g/day/60 kg or more in terms of hydroxycitric acid as a dose to humans. The upper limit of the dose is not particularly limited, and is, for example, 18 g/day/60 kg or less, preferably 10 g/day/60 kg or less, more preferably 2 g/day/60 kg or less, still more preferably 1 g/day/60 kg or less, still more preferably 0.6 g/day/60 kg or less, and still more preferably 0.4 g/day/60 kg or less. In the case of a cartilage regeneration promoter containing a garcinia extract, the dose to humans is, for example, 0.16 g/day/60 kg or more, preferably 0.3 g/day/60 kg or more, and more preferably 0.45 g/day/60 kg or more in terms of dry extract of the garcinia extract. The upper limit of the dose is not particularly limited, and is, for example, 20 g/day/60 kg or less, preferably 10 g/day/60 kg or less, more preferably 5 g/day/60 kg or less, still more preferably 2.5 g/day/60 kg or less, still more preferably 1.2 g/day/60 kg or less, still more preferably 0.8 g/day/60 kg or less, and particularly preferably 0.6 g/day/60 kg or less.

The dose of the cartilage regeneration promoter of the present invention is, as a dose to humans, for example, 1 mg/day/60 kg or more, preferably 2 mg/day/60 kg or more, more preferably 4.5 mg/day/60 kg or more, still more preferably 7 mg/day/60 kg or more, still more preferably 9 mg/day/60 kg or more, particularly preferably 12 mg/day/60 kg or more, 18 mg/day/60 kg or more, or 23 mg/day/60 kg or more in terms of the amount of the component (B). The upper limit of the dose is, for example, 500 mg/day/60 kg or less, preferably 300 mg/day/60 kg or less, more preferably 100 mg/day/60 kg or less, still more preferably 50 mg/day/60 kg or less, still more preferably 45 mg/day/60 kg or less, still more preferably 35 mg/day/60 kg or less, particularly preferably 27 mg/day/60 kg or less, 20 mg/day/60 kg or less, or 16 mg/day/60 kg or less. The dose can be applied regardless of the type of the component (B), but is particularly preferably applied when the component (B) is the component (B1).

The method for administering (ingesting) the cartilage regeneration promoter of the present invention is not particularly limited, but for example, the cartilage regeneration promoter can be administered orally or parenterally once or multiple times a day, and preferably administered orally once or 2 to 3 times a day.

### Other Forms of Cartilage Regeneration Promoter

As described above, the cartilage regeneration promoter of the present invention may be a composition containing the component (A) and the component (B), a combination of the component (A) and the component (B) (for example, a combination form of a composition containing the component (A) and a physically independent composition containing the component (B) can be included), or a composition containing the component (A) and scheduled to be used in combination with the component (B) (for example, a composition containing the component (A) and provided with information indicating that the composition is used in combination with the component (B)). That is, the present invention also provides: a cartilage regeneration promoter made by combining (A) hydroxycitric acid and/or a salt thereof with (B) a sugar selected from a group consisting of chondroitin sulfate and a salt thereof, a proteoglycan, hyaluronic acid and a salt thereof, and a constituent sugar of the above sugars; and a cartilage regeneration promoter including (A) hydroxycitric acid and/or a salt thereof and used to promote cartilage regeneration in combination with (B) a sugar selected from a group consisting of chondroitin sulfate and a salt thereof, a proteoglycan, hyaluronic acid and a salt thereof, and a constituent sugar of the above sugars. Details of the component (A) and the component (B), which are active ingredients in these cartilage regeneration promoters, and details of other optional components, preparation forms, production methods, uses, and doses are as described above.

### 2. Promoter for Differentiation to Cartilaginous Cells

As described above, the combination of the component (A) and the component (B) can promote differentiation from undifferentiated cells to cartilaginous cells, and therefore is also useful as an active ingredient of a promoter for differentiation to cartilaginous cells. Accordingly, the present invention also provides a promoter for differentiation from undifferentiated cells to cartilaginous cells, the promoter including: (A) hydroxycitric acid and/or a salt thereof; and (B) a sugar selected from a group consisting of chondroitin sulfate and a salt thereof, a proteoglycan, hyaluronic acid and a salt thereof, and a constituent sugar of the above sugars.

In the promoter for differentiation to cartilaginous cells, details of the component (A) and the component (B), which are active ingredients, and details of other optional components, preparation forms, production methods, uses, and doses are the same as in the “1. Cartilage regeneration promoter”.

Furthermore, the promoter for differentiation to cartilaginous cells can also be used for the purpose of promoting differentiation to cartilaginous cells by being directly exposed to undifferentiated cells in vitro. In this case, when exposed to undifferentiated cells, the concentration of the promoter for differentiation to cartilaginous cells is not particularly limited, and is a concentration of 0.5 to 30 μM, preferably 1 to 30 μM, more preferably 3 to 30 μM, still more preferably 4.5 to 26 μM, 4.5 to 15 μM, 4.5 to 10 μM, 4.5 to 8 μM, or 4.5 to 6 μM in terms of hydroxycitric acid, for example.

3. Therapeutic Agent for Knee Osteoarthritis, Cartilage Degradation Inhibitor, Protective Agent for Maintaining Cartilage Thickness, and Alleviating Agent for Joint Pain, Joint Uneasiness, and/or Joint Displeasure

As described above, the combination of the component (A) and the component (B) can promote differentiation from undifferentiated cells to cartilaginous cells, and therefore is also useful as an active ingredient of a therapeutic agent for knee osteoarthritis, a cartilage degradation inhibitor, a protective agent for maintaining cartilage thickness, or an alleviating agent for joint pain, joint uneasiness, and/or joint displeasure. Accordingly, the present invention also provides a therapeutic agent for knee osteoarthritis, a cartilage degradation inhibitor, a protective agent for maintaining cartilage thickness, or an alleviating agent for joint pain, joint uneasiness, and/or joint displeasure, each including: (A) hydroxycitric acid and/or a salt thereof; and (B) a sugar selected from a group consisting of chondroitin sulfate and a salt thereof, a proteoglycan, hyaluronic acid and a salt thereof, and a constituent sugar of the above sugars.

In the therapeutic agent for knee osteoarthritis, the cartilage degradation inhibitor, the protective agent for maintaining cartilage thickness, or the alleviating agent for joint pain, joint uneasiness, and/or joint displeasure, details of the component (A) and the component (B), which are active ingredients, and details of other optional components, preparation forms, production methods, uses, and doses are the same as in the “1. Cartilage regeneration promoter”.

### EXAMPLES

Hereinafter, the present invention will be described more specifically with reference to examples, but the present invention is not limited thereto.

### Test Example 1

### Cultured Cells

In accordance with PLOS ONE, (US), 2014, 9 (12), e112291. DOI: 10.1371/journal.pone.0112291 (hereinafter, described as “Document A”), mesenchymal stem cells were induced from human normal iPS cells via neural crest cells, added with an equivalent amount of a cell freezing damage protection liquid CP-1 (manufactured by KYOKUTO PHARMACEUTICAL INDUSTRIAL CO., LTD), and cryopreserved.

### Source of Component (A) (Garcinia Extract)

The pericarp of Garcinia cambogia was dried, and added with 10 times the amount of water and calcium eggshell, and extracted with hot water. The obtained extract was filtered, and then concentrated under reduced pressure using a rotary evaporator, and the obtained concentrated solution was further spray-dried. The dried extract was ground to obtain a powdered garcinia extract. It is to be noted that the garcinia extract contains hydroxycitric acid ((−) hydroxycitric acid) contained in the pericarp of Garcinia cambogia in the form of a calcium salt obtained by using calcium eggshell at the time of extraction. The content of the active ingredient in the garcinia extract is 60 wt % in terms of hydroxycitric acid.

### Component (B)

The following materials were used for blending the component (B).
(B1) Chondroitin sulfate material (porcine cartilage extract; chondroitin sulfate derived from porcine cartilage (the chondroitin sulfate is mainly chondroitin sulfate A) content: 84 wt %)
(B2) Proteoglycan material (salmon (nasal cartilage) extract; proteoglycan derived from salmon (nasal cartilage) content: 22.4 wt %)
(B3) Sodium hyaluronate (derived from cockscomb); In all test examples, including the present test example, it is abbreviated as “HYALURONIC ACID”.
(B4) Glucosamine (derived from shrimp and crab)

### Differentiation to Cartilaginous Cells and Cartilage Matrix Production in Two-Dimensional Culture

Human iPS cell-derived mesenchymal stem cells were thawed and grown, and seeded in a 96 well plate (manufactured by Corning) in 1×10⁵ cells/well. After 24 hours, the culture supernatant was removed, and replaced with a culture medium to induce differentiation to cartilaginous cells in accordance with Document A. To the culture medium to induce differentiation to cartilaginous cells, the garcinia extract and/or the component (B) (components (B1) to (B4)) as test substances were added so that the final concentration was the concentration shown in Tables 1 to 3. No test substance was added to the control group. The cells were cultured for about 10 days, and then fixed with a 4% paraformaldehyde·phosphate buffer (manufactured by FUJIFILM Wako Pure Chemical Corporation). After fixed, the cells were stained with an Alcian blue solution (manufactured by MUTO PURE CHEMICALS CO., LTD.) for 1 hour, and the dye was eluted using a 6 M guanidine hydrochloride solution (manufactured by Tokyo Chemical Industry Co., Ltd.). The eluate was transferred to a 96 well plate, and the OD value of each well at 620 nm was measured with a microplate reader to measure the amount of matrix produced by differentiation-induced cartilaginous cells. Specifically, the average OD values were calculated for each group as the ratio of cartilage matrix production, such that the control group (no test substance, Comparative Example 1) had an average OD value of 1. The results are shown in Tables 1 to 3b.

TABLE 1

| | | Comparative Example | | | | | Example | | |
|---|---|---|---|---|---|---|---|---|---|
| | | 1 | 2 | 3 | 4 | 5 | 1 | 2 | 3 |
| | Garcinia extract [μg/mL] | 0 | 6 | 0 | 0 | 0 | 6 | 6 | 6 |
| (A) | (Amount in extract in terms of | 0 | 3.6 | 0 | 0 | 0 | 3.6 | 3.6 | 3.6 |
| | hydroxycitric acid [μg/mL]) | | | | | | | | |
| | Porcine cartilage extract | 0 | 0 | 0.05 | 0.19 | 0.38 | 0.05 | 0.19 | 0.38 |
| | [μg/mL] | | | | | | | | |
| (B1) | (Chondroitin sulfate in | 0 | 0 | 0.04 | 0.16 | 0.32 | 0.04 | 0.16 | 0.32 |
| | extract [μg/mL]) | | | | | | | | |
| | (B1)/Garcinia extract | | 0 | | | | 0.006 | 0.027 | 0.053 |
| | (B1)/(A) | | 0 | | | | 0.011 | 0.044 | 0.088 |
| | Ratio of cartilage matrix | 1.00 | 1.05 | 1.01 | 1.01 | 1.01 | 1.12 | 1.24 | 1.27 |
| | production | | | | | | | | |

TABLE 2a

| | | Comparative Example | | | | | | | | | | |
|---|---|---|---|---|---|---|---|---|---|---|---|---|
| | | 1 | 2 | 6 | 7 | 8 | 9 | 10 | 11 | 12 | 13 | 14 |
| | Garcinia extract [μg/mL] | 0 | 6 | 0 | 0 | 0 | 0 | 0 | 0 | 0 | 0 | 0 |
| (A) | (Amount in extract in terms of | 0 | 3.6 | 0 | 0 | 0 | 0 | 0 | 0 | 0 | 0 | 0 |
| | hydroxycitric acid [μg/mL]) | | | | | | | | | | | |
| (B4) | Glucosamine [μg/mL] | 0 | 0 | 0.006 | 0.045 | 0.094 | 0.190 | 0.375 | 0.750 | 1.500 | 3.000 | 6.000 |
| | (B4)/Garcinia extract | | 0 | | | | | | | | | |
| | (B4)/(A) | | 0 | | | | | | | | | |
| | Ratio of cartilage matrix | 1.00 | 1.05 | 1.00 | 1.01 | 1.01 | 1.01 | 1.01 | 1.01 | 1.02 | 1.00 | 1.00 |
| | production | | | | | | | | | | | |

TABLE 2b

| | | Example | | | | | | | | |
|---|---|---|---|---|---|---|---|---|---|---|
| | | 4 | 5 | 6 | 7 | 8 | 9 | 10 | 11 | 12 |
| | Garcinia extract [μg/mL] | 6 | 6 | 6 | 6 | 6 | 6 | 6 | 6 | 6 |
| (A) | (Amount in extract in terms of | 3.6 | 3.6 | 3.6 | 3.6 | 3.6 | 3.6 | 3.6 | 3.6 | 3.6 |
| | hydroxycitric acid [μg/mL]) | | | | | | | | | |
| (B4) | Glucosamine [μg/mL] | 0.006 | 0.045 | 0.094 | 0.190 | 0.375 | 0.750 | 1.500 | 3.000 | 6.000 |
| | (B4)/Garcinia extract | 0.001 | 0.01 | 0.02 | 0.03 | 0.06 | 0.13 | 0.25 | 0.5 | 1 |
| | (B4)/(A) | 0.0017 | 0.013 | 0.026 | 0.053 | 0.10 | 0.21 | 0.42 | 0.83 | 1.7 |
| | Ratio of cartilage matrix | 1.10 | 1.13 | 1.15 | 1.19 | 1.21 | 1.24 | 1.15 | 1.11 | 1.10 |
| | production | | | | | | | | | |

TABLE 2c

| | | Comparative Example | | | | | | |
|---|---|---|---|---|---|---|---|---|
| | | 1 | 2 | 15 | 16 | 17 | 18 | 19 |
| | Garcinia extract [μg/mL] | 0 | 6 | 0 | 0 | 0 | 0 | 0 |
| (A) | (Hydroxycitric acid in extract | 0 | 3.6 | 0 | 0 | 0 | 0 | 0 |
| | [μg/mL]) | | | | | | | |
| (B3) | Hyaluronic acid [μg/mL] | 0 | 0 | 0.006 | 0.06 | 0.6 | 6 | 60 |
| | (B3)/Garcinia extract | | 0 | | | | | |
| | (B3)/(A) | | 0 | | | | | |
| | Ratio of cartilage matrix | 1.00 | 1.05 | 1.01 | 1.01 | 1.02 | 1.02 | 1.02 |
| | production | | | | | | | |

TABLE 2d

| | | Example | | | | |
|---|---|---|---|---|---|---|
| | | 13 | 14 | 15 | 16 | 17 |
| | Garcinia extract [μg/mL] | 6 | 6 | 6 | 6 | 6 |
| (A) | (Hydroxycitric acid in extract | 3.6 | 3.6 | 3.6 | 3.6 | 3.6 |
| | [μg/mL]) | | | | | |
| (B3) | Hyaluronic acid [μg/mL] | 0.006 | 0.06 | 0.6 | 6 | 60 |
| | (B3)/Garcinia extract | 0 | 0.01 | 0.1 | 1 | 10 |
| | (B3)/(A) | 0.002 | 0.017 | 0.167 | 1.667 | 16.67 |
| | Ratio of cartilage matrix | 1.11 | 1.14 | 1.17 | 1.15 | 1.11 |
| | production | | | | | |

TABLE 3a

| | | Comparative Example | | | | | | | | | | |
|---|---|---|---|---|---|---|---|---|---|---|---|---|
| | | 1 | 2 | 20 | 21 | 22 | 23 | 24 | 25 | 26 | 27 | 28 |
| | Garcinia extract [μg/mL] | 0 | 6 | 0 | 0 | 0 | 0 | 0 | 0 | 0 | 0 | 0 |
| (A) | (Amount in extract in terms of | 0 | 3.6 | 0 | 0 | 0 | 0 | 0 | 0 | 0 | 0 | 0 |
| | hydroxycitric acid [μg/mL]) | | | | | | | | | | | |
| | Salmon cartilage extract | 0 | 0 | 0.006 | 0.045 | 0.094 | 0.190 | 0.375 | 0.750 | 1.500 | 3.000 | 6.000 |
| | [μg/mL] | | | | | | | | | | | |
| (B2) | (Proteoglycan in extract | 0 | 0 | 0.001 | 0.010 | 0.021 | 0.043 | 0.084 | 0.168 | 0.336 | 0.672 | 1.34 |
| | [μg/mL]) | | | | | | | | | | | |
| | (B2)/Garcinia extract | | 0 | | | | | | | | | |
| | (B2)/(A) | | 0 | | | | | | | | | |
| | Ratio of cartilage matrix | 1.00 | 1.05 | 1.00 | 1.01 | 1.02 | 1.02 | 1.02 | 1.01 | 1.01 | 1.01 | 1.00 |
| | production | | | | | | | | | | | |

TABLE 3b

| | | Example | | | | | | | | |
|---|---|---|---|---|---|---|---|---|---|---|
| | | 18 | 19 | 20 | 21 | 22 | 23 | 24 | 25 | 26 |
| | Garcinia extract [μg/mL] | 6 | 6 | 6 | 6 | 6 | 6 | 6 | 6 | 6 |
| (A) | (Amount in extract in terms of | 3.6 | 3.6 | 3.6 | 3.6 | 3.6 | 3.6 | 3.6 | 3.6 | 3.6 |
| | hydroxycitric acid [μg/mL]) | | | | | | | | | |
| | Salmon cartilage extract | 0.006 | 0.045 | 0.094 | 0.190 | 0.375 | 0.750 | 1.500 | 3.000 | 6.000 |
| | [μg/mL] | | | | | | | | | |
| (B2) | (Proteoglycan in extract | 0.001 | 0.010 | 0.021 | 0.043 | 0.084 | 0.168 | 0.336 | 0.672 | 1.34 |
| | [μg/mL]) | | | | | | | | | |
| | (B2)/Garcinia extract | 0.00022 | 0.0017 | 0.004 | 0.01 | 0.01 | 0.03 | 0.06 | 0.11 | 0.22 |
| | (B2)/(A) | 0.00037 | 0.003 | 0.006 | 0.012 | 0.023 | 0.047 | 0.093 | 0.187 | 0.373 |
| | Ratio of cartilage matrix | 1.11 | 1.12 | 1.18 | 1.25 | 1.24 | 1.23 | 1.18 | 1.15 | 1.10 |
| | production | | | | | | | | | |

As shown in Tables 1 to 3b, the combination between a garcinia extract and chondroitin sulfate, glucosamine, hyaluronic acid, or a proteoglycan remarkably improved the ratio of cartilage matrix production. This also indicates that prior to cartilage matrix regeneration, the combination between a garcinia extract and chondroitin sulfate, glucosamine, hyaluronic acid, or a proteoglycan remarkably induced differentiation of human iPS cell-derived mesenchymal stem cells into cartilaginous cells to produce cartilage matrix.

### Test Example 2

### Cultured Cells

In accordance with Tissue Engineering, (US), 2010, 16 (1), p 81-91. DOI: 10.1089=ten.tec.2008.0693, mesenchymal stem cells were isolated from human normal bone marrow in a dedicated culture medium, and then the cells were grown. The grown cells were recovered, added with an equivalent amount of a cell freezing damage protection liquid CP-1 (KYOKUTO PHARMACEUTICAL INDUSTRIAL CO., LTD), and cryopreserved.

### Source of Component (A)

The garcinia extract prepared in Test example 1 was used.

### Component (A)

(−) Hydroxycitric acid-3 calcium (HCA·Ca) (manufactured by Sigma-Aldrich Co. LLC)·

### Source of Component (B)

The same material as those used in Test example 1 was used.

### Induction of Differentiation to Cartilaginous Cells in Three-Dimensional Culture

It is known that cartilaginous cells dedifferentiate in two-dimensional culture. As a culture system of cartilaginous cells closer to nature, a technique called three-dimensional culture is adopted. The thawed and grown human bone marrow-derived mesenchymal stem cells were dispensed into 15 mL tubes (made of TPP) by 2.5×10⁵ cells, and centrifuged at 1000 rpm for 3 minutes at room temperature. The supernatant was removed and replaced with 0.5 mL of a culture medium to induce differentiation to cartilaginous cells in accordance with Document A. At the same time, as test substances, the garcinia extract, HCA·Ca, and/or the component (B) (components (B1) to (B4)) were added at the concentration shown in Tables 4 to 11. After cultured for 21 days, the cells were fixed in a 4% paraformaldehyde·phosphate buffer for 12 hours, dehydrated in 70% ethanol, and then embedded in paraffin, and then specimens were prepared. In the prepared specimen, the acidic polysaccharides as a cartilage matrix was stained with safranin O, and then the morphology of differentiated cartilaginous cells was observed using an optical microscope (magnification: 10 times). Furthermore, regarding the area showing a red color by safranin O staining, the relative area (differentiated cartilaginous cell area ratio) was calculated using an image analysis software Image J such that the control (Comparative Example 1) had the area of 1. The results are shown in Tables 4 to 11.

TABLE 4

| | | Comparative Example | | | | Example | | | |
|---|---|---|---|---|---|---|---|---|---|
| | | 1 | 29 | 30 | 31 | 27 | 28 | 29 | 30 |
| | Garcinia extract [μg/mL] | 0 | 5 | 0 | 0 | 5 | 5 | 5 | 5 |
| (A) | (Amount in extract in terms of | 0 | 3 | 0 | 0 | 3 | 3 | 3 | 3 |
| | hydroxycitric acid [μg/mL]) | | | | | | | | |
| | Porcine cartilage extract | 0 | 0 | 0.5 | 5 | 0.005 | 0.05 | 0.5 | 5 |
| | [μg/mL] | | | | | | | | |
| (B1) | (Chondroitin sulfate in extract | 0 | 0 | 0.42 | 4.2 | 0.0042 | 0.042 | 0.42 | 4.2 |
| | [μg/mL]) | | | | | | | | |
| | (B1)/Garcinia extract | | 0 | | | 0.00084 | 0.0084 | 0.084 | 0.84 |
| | (B1)/(A) | | 0 | | | 0.0014 | 0.014 | 0.14 | 1.4 |
| | Differentiated cartilaginous cell | 1 | 1.8 | 0.1 | 0.9 | 8.3 | 16.1 | 16.8 | 4.2 |
| | area ratio | | | | | | | | |

TABLE 5

| | | Comparative Example | | | | Example | | |
|---|---|---|---|---|---|---|---|---|
| | | 1 | 32 | 30 | 31 | 31 | 32 | 33 |
| (A) | HCA•Ca [μg/mL] | 0 | 5 | 0 | 0 | 5 | 5 | 5 |
| | (Amount in terms of | 0 | 3.92 | 0 | 0 | 3.92 | 3.92 | 3.92 |
| | hydroxycitric acid [μg/mL]) | | | | | | | |
| | Porcine cartilage extract | 0 | 0 | 0.5 | 5 | 0.005 | 0.5 | 5 |
| | [μg/mL] | | | | | | | |
| (B1) | (Chondroitin sulfate in extract | 0 | 0 | 0.42 | 4.2 | 0.0042 | 0.42 | 4.2 |
| | [μg/mL]) | | | | | | | |
| | (B1)/(A) Amount in terms of | | 0 | | | 0.0011 | 0.11 | 1.1 |
| | hydroxycitric acid | | | | | | | |
| | Differentiated cartilaginous cell | 1 | 1.7 | 0.1 | 0.9 | 12.7 | 40.6 | 7.2 |
| | area ratio | | | | | | | |

TABLE 6

| | | Comparative Example | | | | Example | | | |
|---|---|---|---|---|---|---|---|---|---|
| | | 1 | 29 | 33 | 34 | 34 | 35 | 36 | 37 |
| | Garcinia extract [μg/mL] | 0 | 5 | 0 | 0 | 5 | 5 | 5 | 5 |
| (A) | (Amount in extract in terms of | 0 | 3 | 0 | 0 | 3 | 3 | 3 | 3 |
| | hydroxycitric acid [μg/mL]) | | | | | | | | |
| (B4) | Glucosamine [μg/mL] | 0 | 0 | 0.5 | 5 | 0.005 | 0.05 | 0.5 | 5 |
| | (B4)/Garcinia extract | | 0 | | | 0.001 | 0.01 | 0.1 | 1 |
| | (B4)/(A) | | 0 | | | 0.00167 | 0.0167 | 0.167 | 1.67 |
| | Differentiated cartilaginous cell | 1 | 1.8 | 0.1 | 0.2 | 5.3 | 5.5 | 9.4 | 2.3 |
| | area ratio | | | | | | | | |

TABLE 7

| | | Comparative Example | | | | Example | |
|---|---|---|---|---|---|---|---|
| | | 1 | 32 | 33 | 34 | 38 | 39 |
| (A) | HCA•Ca [μg/mL] | 0 | 5 | 0 | 0 | 5 | 5 |
| | (Amount in terms of | 0 | 3.92 | 0 | 0 | 3.92 | 3.92 |
| | hydroxycitric acid [μg/mL]) | | | | | | |
| (B4) | Glucosamine [μg/mL] | 0 | 0 | 0.5 | 5 | 0.5 | 5 |
| | (B4)/(A) Amount in terms of | | 0 | | | 0.127 | 1.27 |
| | hydroxycitric acid | | | | | | |
| | Differentiated cartilaginous cell | 1 | 1.7 | 0.1 | 0.2 | 36.4 | 4.7 |
| | area ratio | | | | | | |

TABLE 8

| | | Comparative Example | | | | Example | |
|---|---|---|---|---|---|---|---|
| | | 1 | 29 | 35 | 36 | 40 | 41 |
| | Garcinia extract [μg/mL] | 0 | 5 | 0 | 0 | 5 | 5 |
| (A) | (Amount in extract in terms of | 0 | 3 | 0 | 0 | 3 | 3 |
| | hydroxycitric acid [μg/mL]) | | | | | | |
| (B3) | Hyaluronic acid [μg/mL] | 0 | 0 | 0.5 | 5 | 0.5 | 5 |
| | (B3)/(A) | | 0 | | | 0.167 | 1.67 |
| | Differentiated cartilaginous cell | 1 | 3.5 | 1.4 | 1.7 | 11.4 | 8.7 |
| | area ratio | | | | | | |

TABLE 9

| | | Comparative Example | | | | Example | |
|---|---|---|---|---|---|---|---|
| | | 1 | 32 | 35 | 36 | 42 | 43 |
| (A) | HCA•Ca [μg/mL] | 0 | 5 | 0 | 0 | 5 | 5 |
| | (Amount in terms of | 0 | 3.92 | 0 | 0 | 3.92 | 3.92 |
| | hydroxycitric acid [μg/mL]) | | | | | | |
| (B3) | Hyaluronic acid [μg/mL] | 0 | 0 | 0.5 | 5 | 0.5 | 5 |
| | (B3)/(A) Amount in terms of | | 0 | | | 0.127 | 1.27 |
| | hydroxycitric acid | | | | | | |
| | Differentiated cartilaginous cell | 1 | 3.5 | 1.4 | 1.7 | 17.2 | 8.8 |
| | area ratio | | | | | | |

TABLE 10

| | | Comparative Example | | | | Example | |
|---|---|---|---|---|---|---|---|
| | | 1 | 29 | 37 | 38 | 44 | 45 |
| | Garcinia extract [μg/mL] | 0 | 5 | 0 | 0 | 5 | 5 |
| (A) | (Amount in extract in terms of | 0 | 3 | 0 | 0 | 3 | 3 |
| | hydroxycitric acid [μg/mL]) | | | | | | |
| | Salmon cartilage extract | 0 | 0 | 0.5 | 5 | 0.005 | 0.05 |
| | [μg/mL] | | | | | | |
| (B2) | (Proteoglycan in extract | 0 | 0 | 0.11 | 1.1 | 0.0011 | 0.011 |
| | [μg/mL]) | | | | | | |
| | (B2)/Garcinia extract | | 0 | | | 0.0002 | 0.0022 |
| | (B2)/(A) | | 0 | | | 0.00037 | 0.0037 |
| | Differentiated cartilaginous cell | 1 | 2.1 | 0.2 | 0.5 | 4.4 | 7.6 |
| | area ratio | | | | | | |

TABLE 11

| | | Comparative Example | | | | Example | | |
|---|---|---|---|---|---|---|---|---|
| | | 1 | 32 | 37 | 38 | 46 | 47 | 48 |
| (A) | HCA•Ca [μg/mL] | 0 | 5 | 0 | 0 | 5 | 5 | 5 |
| | (Amount in terms of | 0 | 3.92 | 0 | 0 | 3.92 | 3.92 | 3.92 |
| | hydroxycitric acid [μg/mL]) | | | | | | | |
| | Salmon cartilage extract | 0 | 0 | 0.5 | 5 | 0.005 | 0.05 | 5 |
| | [μg/mL] | | | | | | | |
| (B2) | (Proteoglycan in extract | 0 | 0 | 0.11 | 1.1 | 0.0011 | 0.011 | 1.1 |
| | [μg/mL]) | | | | | | | |
| | (B2)/(A) Amount in terms of | | 0 | | | 0.00029 | 0.0029 | 0.29 |
| | hydroxycitric acid | | | | | | | |
| | Differentiated cartilaginous cell | 1 | 1.5 | 0.2 | 0.5 | 3.2 | 9 | 3 |
| | area ratio | | | | | | | |

As is clear from Tables 4 to 11, the following was observed. The garcinia extract or calcium hydroxycitrate alone exhibits an activity to induce differentiation to cartilaginous cells, the degree of which is limited. In addition, hyaluronic acid alone exhibits almost no activity to induce differentiation to cartilaginous cells, and chondroitin sulfate, glucosamine, or a proteoglycan alone reduce the activity to induce differentiation to cartilaginous cells, rather than not having the activity. However, the combination between the garcinia extract or calcium hydroxycitrate and chondroitin sulfate, glucosamine, hyaluronic acid, or a proteoglycan remarkably promotes the activity to induce differentiation to cartilaginous cells.

### Cartilage Matrix Production Activity

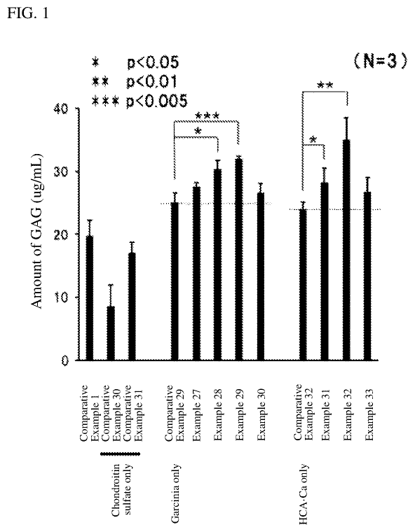

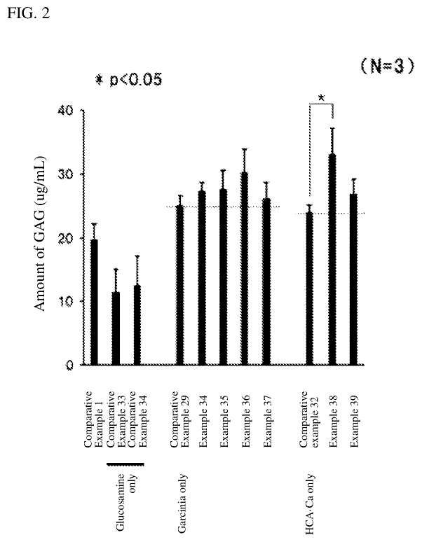

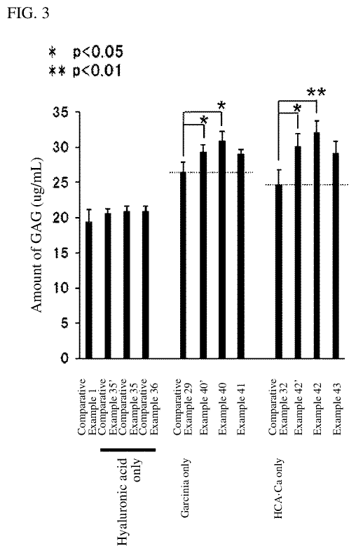

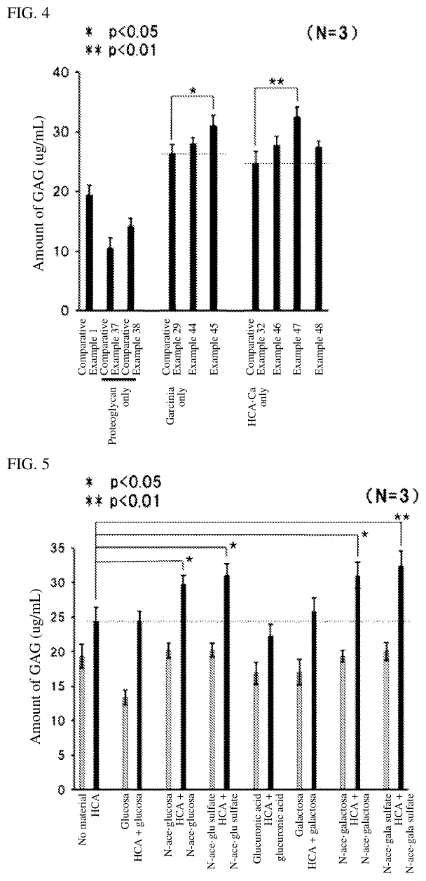

The cartilaginous cell masses (pellets) obtained in Comparative Examples and Examples in Tables 4 to 11 were decomposed with a digestion enzyme papain solution (papain manufactured by Sigma-Aldrich Co. LLC is solubilized in 100 mM phosphate buffer pH 7.4, 5 mM L-cysteine hydrochloride monohydrate, and 10 mM EDTA), and then the amount of GAG (glycosaminoglycan) was measured using a GAG quantification kit (manufactured by Blyscan). The larger the amount of GAG, the larger the amount of cartilage matrix produced. The results are shown in FIGS. 1 to 4. (in FIG. 3, Comparative Example 35′, Example 40′, and Example 42′ represent the results of the same operation as in Comparative Example 35, Example 40, and Example 42, respectively, except that the hyaluronic acid content was changed to 0.05 μg/mL)

As shown in FIGS. 1 to 4, the following was observed. The garcinia extract or calcium hydroxycitrate alone exhibits a cartilage matrix production activity, the degree of which is limited. In addition, hyaluronic acid alone exhibits almost no activity to induce differentiation to cartilaginous cells, and chondroitin sulfate, glucosamine, or a proteoglycan alone reduce the cartilage matrix production activity, rather than not having the activity. However, the combination between the garcinia extract or calcium hydroxycitrate and chondroitin sulfate, glucosamine, hyaluronic acid, or a proteoglycan remarkably promotes the cartilage matrix production activity.

### Test Example 3

Except that the component (component (B4)) shown in Table 12 (a) and Table 12 (b) was used as the component (B), and the addition amount of the component (A) and the component (B) (component (B4)) was as shown in Table 12 (a) and Table 12 (b), the same procedure as in Test example 2 was carried out to calculate the differentiated cartilaginous cell area ratio and measure the amount of GAG. The results of the differentiated cartilaginous cell area ratio are shown in Table 12 (a) and Table 12 (b), and the measurement results of the amount of GAG are shown in FIG. 5.

The details of the component (B4) used in this test example are as follows.
D-(+)-glucosamine hydrochloride (manufactured by Sigma-Aldrich Co. LLC)
In FIG. 5, it is abbreviated as “GLUCOSA”.
N-acetyl-D-glucosamine (manufactured by Sigma-Aldrich Co. LLC)
In FIG. 5, it is abbreviated as “N-ACE·GLUCOSA”.
Sodium N-acetyl-D-glucosamine 6-sulfate (manufactured by Sigma-Aldrich Co. LLC)
In FIG. 5, it is abbreviated as “N-ACE·GLU SULFATE”.
D-glucuronic acid (manufactured by Sigma-Aldrich Co. LLC)
D-(+)-galactosamine hydrochloride (manufactured by Sigma-Aldrich Co. LLC)
In FIG. 5, it is abbreviated as “GALACTOSA”.
N-acetyl-D-galactosamine (manufactured by Sigma-Aldrich Co. LLC)
In FIG. 5, it is abbreviated as “N-ACE·GALACTOSA”.
Sodium N-acetyl-D-galactosamine 6-sulfate (manufactured by Sigma-Aldrich Co. LLC)
In FIG. 5, it is abbreviated as “N-ACE·GALA SULFATE”.

TABLE 12a

| | | Comparative Example | | | | | | | | |
|---|---|---|---|---|---|---|---|---|---|---|
| | | 1 | 39 | 40 | 41 | 42 | 43 | 44 | 45 | 46 |
| (A) | HCA•Ca | — | 25 | — | — | — | — | — | — | — |
| | (Amount in terms of | — | 50 | — | — | — | — | — | — | — |
| | hydroxycitric acid) | | | | | | | | | |
| (B4) | Glucosamine hydrochloride | — | — | 2.5 | — | — | — | — | — | — |
| | N-acetylglucosamine | — | — | — | 2.5 | — | — | — | — | — |
| | Na N-acetylglucosamine 6- | — | — | — | — | 2.5 | — | — | — | — |
| | sulfate | | | | | | | | | |
| | Glucuronic acid | — | — | — | — | — | 2.5 | — | — | — |
| | Galactosamine hydrochloride | — | — | 2 | — | — | — | 2.5 | — | — |
| | N-acetylgalactosamine | — | — | — | — | — | — | — | 2.5 | — |
| | Na N-acetylgalactosamine 6- | — | — | — | — | — | — | — | — | 2.5 |
| | sulfate | | | | | | | | | |
| | (B4)/(A) | | 0 | | | | | | | |
| | Differentiated cartilaginous cell | 1 | 1.8 | 0.6 | 1.8 | 2 | 0.7 | 0.7 | 1 | 1.4 |
| | area ratio | | | | | | | | | |

In the table, for each of the numerical values indicating the blended amount of the Components, the unit is μM.
In the ratio of (B4)/(A), (A) is an amount in terms of hydroxycitric acid.
The ratio of (B4)/(A) is in weight ratio.

TABLE 12b

| | | Example | | | | | | |
|---|---|---|---|---|---|---|---|---|
| | | 49 | 50 | 51 | 52 | 53 | 54 | 55 |
| (A) | HCA•Ca | 25 | 25 | 25 | 25 | 25 | 25 | 25 |
| | (Amount in terms of | 50 | 50 | 50 | 50 | 50 | 50 | 50 |
| | hydroxycitric acid) | | | | | | | |
| (B4) | Glucosamine hydrochloride | 2.5 | — | — | — | — | — | — |
| | N-acetylglucosamine | — | 2.5 | — | — | — | — | — |
| | Na N-acetylglucosamine 6- | — | — | 2.5 | — | — | — | — |
| | sulfate | | | | | | | |
| | Glucuronic acid | — | — | — | 2.5 | — | — | — |
| | Galactosamine hydrochloride | — | — | — | — | 2.5 | — | — |
| | N-acetylgalactosamine | — | — | — | — | — | 2.5 | — |
| | Na N-acetylgalactosamine 6- | — | — | — | — | — | — | 2.5 |
| | sulfate | | | | | | | |
| | (B4)/(A) | 0.05 | 0.05 | 0.1 | 0.05 | 0.05 | 0.05 | 0.1 |
| | Differentiated cartilaginous cell | 4.6 | 9.8 | 11.3 | 2.3 | 6.9 | 10.7 | 16.1 |
| | area ratio | | | | | | | |

In the table, for each of the numerical values indicating the blended amount of the Components, the unit is μM.
In the ratio of (B4)/(A), (A) is an amount in terms of hydroxycitric acid.
The ratio of (B4)/(A) is in weight ratio.

As is clear from Table 12 (a), Table 12 (b), and FIG. 5, the following was observed. The component (A) alone exhibits an activity to induce differentiation to cartilaginous cells, the degree of which is limited. In addition, the component (B4) alone reduces the activity to induce differentiation to cartilaginous cells or only exhibits an activity to induce differentiation to cartilaginous cells. However, the combination between the component (A) and the component (B4) remarkably promotes the activity to induce differentiation to cartilaginous cells. Furthermore, the following was found. When an optionally N-acetylated hexosamine, such as glucosamine, N-acetylglucosamine, sodium N-acetylglucosamine 6-sulfate, galactosamine, N-acetylgalactosamine, or sodium N-acetylgalactosamine sulfate, is used as the component (B4), the activity to induce differentiation to cartilaginous cells is highly promoted. Especially, when N-acetylgalactosamine, or sodium N-acetylgalactosamine 6-sulfate is used as the component (B4), the activity to induce differentiation to cartilaginous cells is more highly promoted.

### Test Example 4

### Test Subject

In accordance with Osteoarthritis Cartilage, 2012, 20 (8), 887-895. DOI: 10.1016/j.joca.2012.04.012, mice were forced to walk for 2 weeks on a 15 degree uphill under conditions of 20 m/min, 20 min/day using a treadmill apparatus (belt conveyor, Muromachi Kikai Co., Ltd.) to promote degeneration of knee cartilage. Thus, an animal experimental model was constructed without surgery, the model reproducing human age-related cartilage degeneration symptoms (knee osteoarthritis), that is, knee cartilage attrition symptoms.

### Source of Component (A)

The garcinia extract prepared in Test example 1 was used.

### Source of Component (B1)

The same chondroitin sulfate as used in Test example 1 was used.

### Test Schedule and Test Method

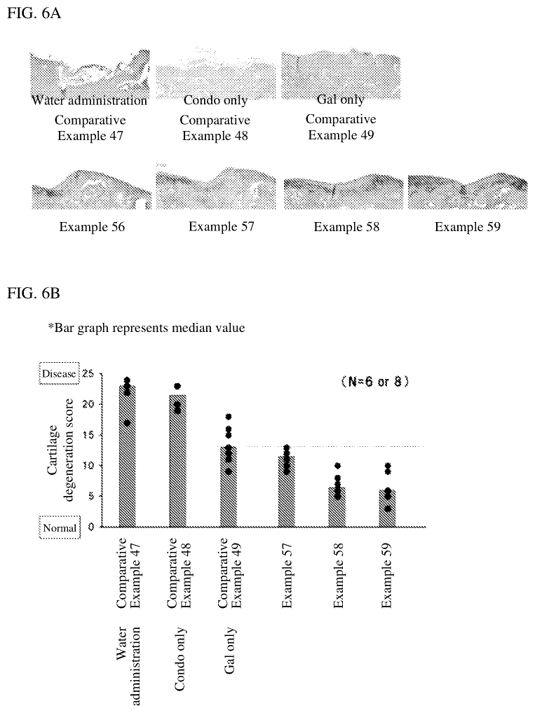

The garcinia extract and/or chondroitin sulfate were orally administered at doses shown in Table 13 for 8 weeks. The garcinia extract dose of 102.5 mg/day/kg corresponds to 0.5 g/day in terms of human equivalent dose (using a divisor of 12.3 for mice and assuming that a human is 60 kg), and the dose of 61.5 mg/day/kg in terms of hydroxycitric acid corresponds to 0.3 g/day in terms of human equivalent dose (using a divisor of 12.3 for mice and assuming that a human is 60 kg). After the 8-week administration was completed, the mice were dissected and the blood was collected. Then specimens stained with safranin O were prepared for the articular cartilage tissue of the right femur. The results of the tissue specimen analysis are shown in FIG. 6A. Furthermore, based on the stained tissue specimens, cartilage degeneration was evaluated by scoring (Gerwin et al. Osteoarthritis Cartilage. 2010 October; 18 Suppl 3: S24-34). The results are shown in FIG. 6B. In FIGS. 6A and 6B, chondroitin sulfate is abbreviated as “CONDO”, and the garcinia extract is abbreviated as “GAL”.

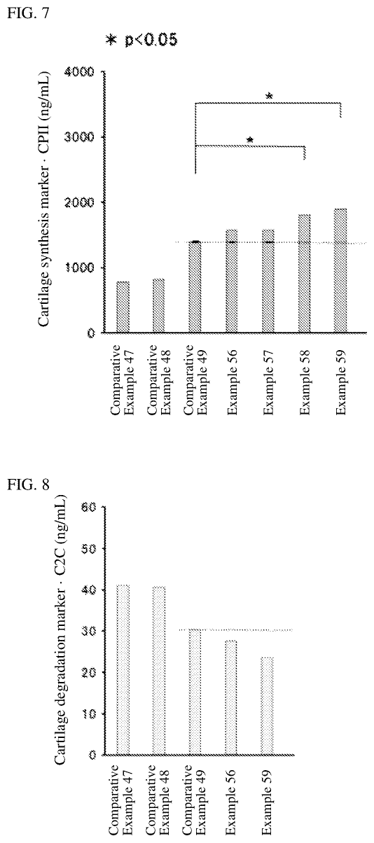

In addition, blood was collected from each of the mice at the time of dissection to prepare serum, and the cartilage synthesis marker CPII and the cartilage degradation marker C2C were quantified using a commercially available kit (IBEX). The measurement result of the cartilage synthesis marker CPII is shown in FIG. 7, and the measurement result of the cartilage degradation marker C2C is shown in FIG. 8.

TABLE 13

| | | Comparative Example | | | Example | | | |
|---|---|---|---|---|---|---|---|---|
| | | 47 | 48 | 49 | 56 | 57 | 58 | 59 |
| | Dose of garcinia extract | 0 | 0 | 102.5 | 102.5 | 102.5 | 102.5 | 102.5 |
| | [mg/day/kg] | | | | | | | |
| (A) | (Amount in extract in terms of | 0 | 0 | 61.5 | 61.5 | 61.5 | 61.5 | 61.5 |
| | hydroxycitric acid [mg/day/kg]) | | | | | | | |
| | Dose of porcine cartilage | 0 | 102.5 | 0 | 1.025 | 2.05 | 3.075 | 5.125 |
| | extract [mg/day/kg] | | | | | | | |
| (B1) | (Dose of chondroitin sulfate in | 0 | 86.1 | 0 | 0.861 | 1.722 | 2.583 | 4.305 |
| | extract [mg/day/kg]) | | | | | | | |
| | (B1)/Garcinia extract | | | 0 | 0.008 | 0.017 | 0.025 | 0.042 |
| | (B1)/(A) | | | 0 | 0.014 | 0.028 | 0.042 | 0.07 |
| | N | 8 | 6 | 8 | 8 | 8 | 8 | 8 |

### Results

As is clear from FIG. 6A, when hydroxycitric acid and chondroitin sulfate were administered in combination (Examples 56 to 59), a staining pattern showing formation of hyaline cartilage and remarkable regeneration of articular cartilage tissue was observed, which was not observed when the garcinia extract alone was administered (Comparative Example 49) or chondroitin sulfate alone was administered (Comparative Example 48). Especially, when both components were combined in the ratio of Examples 58 and 59, a staining pattern showing an especially remarkable regeneration was observed. The results shown in FIG. 6A were also reflected in the results of FIG. 6B.

In addition, as is clear from FIG. 7, the following was observed. The garcinia extract alone exhibits the effect of increase in the cartilage synthesis marker, the degree of which is limited (Comparative Example 49), and chondroitin sulfate alone exhibits almost no increase in the cartilage synthesis marker even at a high dose (Comparative Example 48). However, only by combining a small amount of chondroitin sulfate with the garcinia extract, the cartilage synthesis marker is remarkably increased (Examples 56 to 59), and the result was especially remarkable in the combination of both components in the ratio of Examples 58 and 59.

In addition, as is clear from FIG. 8, the following was observed. The garcinia extract alone exhibits the effect of decrease in the cartilage degradation marker, the degree of which is limited (Comparative Example 49), and chondroitin sulfate alone exhibits no effect of decrease in the cartilage degradation marker even at a high dose (Comparative Example 48). However, only by combining a small amount of chondroitin sulfate with the garcinia extract, the cartilage degradation marker is remarkably decreased (Examples 56 and 59), which reflects a tendency of the inverse relationship with FIG. 7 regarding the cartilage synthesis marker.

Stem Cells International, 2017, Article ID 9312329, DOI: 10.1155/2017/9312329 proves that mesenchymal stem cells (MSC) are present in the synovial and joint fluid of human's knee joint and hip joint. In the model for promoting knee joint cartilage degeneration by forcing a mouse to walk without surgery, it is considered that MSCs in the synovial and the joint fluid were differentiated into cartilaginous cells by administering the combination of the garcinia extract and chondroitin sulfate, and thus cartilage tissue regeneration was shown. From the above, it is considered that cartilage tissue regeneration is promoted by intake of the combination of a garcinia extract and chondroitin sulfate even in age-related natural cartilage abrasion in humans.

1. A cartilage regeneration promoter comprising: (A) hydroxycitric acid and/or a salt of hydroxycitric acid; and (B) a sugar selected from a group consisting of chondroitin sulfate and a salt of chondroitin sulfate, a proteoglycan, hyaluronic acid and a salt of hyaluronic acid, and a constituent sugar of the above sugars.

2. The cartilage regeneration promoter according to claim 1, wherein the constituent sugar is an optionally N-acetylated hexosamine selected from a group consisting of glucosamine, N-acetylglucosamine, N-acetylglucosamine sulfate and a salt of N-acetylglucosamine sulfate, galactosamine, N-acetylgalactosamine, and N-acetylgalactosamine sulfate and a salt of N-acetylgalactosamine sulfate.

3. The cartilage regeneration promoter according to claim 1, wherein the component (B) is contained in a total amount of 0.001 to 6 parts by weight with respect to 1 part by weight of the component (A) in a total amount in terms of hydroxycitric acid.

4. The cartilage regeneration promoter according to claim 1, including a plant extract containing the component (A).

5. The cartilage regeneration promoter according to claim 4, wherein the plant extract is a garcinia extract.

6. The cartilage regeneration promoter according to claim 4, wherein the component (B) is contained in a total amount of 0.0015 to 10 parts by weight with respect to 1 part by weight of the plant extract.

7. The cartilage regeneration promoter according to claim 1, wherein the component (B) is chondroitin sulfate.

8. A method of promoting for differentiation from undifferentiated cells to cartilaginous cells, the method comprising administering the cartilage regeneration promoter according to claim 1 to the undifferentiated cells.

9. A method of treating knee osteoarthritis comprising administering the cartilage regeneration promoter according to claim 1 to a subject.

10. A method of inhibiting cartilage degradation inhibitor comprising administering the cartilage regeneration promoter according to claim 1 to cartilage.

11. A method of maintaining cartilage thickness comprising administering the cartilage regeneration promoter according to claim 1 to cartilage.

12. An method of alleviating joint pain, joint uneasiness, and/or joint displeasure comprising administering the cartilage regeneration promoter according to claim 1 to a subject.
